(19) United States
(12) Patent Application Publication
(10) Pub. No.: US 2024/0119623 A1
(43) Pub. Date: Apr. 11, 2024

(54) POSTURE ANALYSIS SYSTEM USING SMART MIRROR AND CAMERA

(71) Applicant: MDA Co., Ltd, Daegu (KR)

(72) Inventors: Jin Wook CHOI, Daegu (KR); Kyoung Dong KIM, Daegu (KR); Mi Jin KIM, Gumi-si (KR)

(21) Appl. No.: 18/472,005

(22) Filed: Sep. 21, 2023

(30) Foreign Application Priority Data
Oct. 7, 2022 (KR) 10-2022-0128476

(51) Int. Cl. G06T 7/70; G06F 3/01; G06T 7/60; G06V 40/20

(52) U.S. Cl. CPC G06T 7/70; G06F 3/013; G06T 7/60; G06V 40/25; G06V 40/28; G06V 2201/07

(57) ABSTRACT

There is disclosed a posture analysis system using a smart mirror and a camera. The posture analysis system includes a smart mirror configured to reflect a posture of a user, a camera module configured to photograph the posture of the user and generate a posture image, a posture recognition module configured to recognize the posture image generated by the camera module, an image storage control module configured to automatically perform control to store the posture image, an image storage module configured to store the posture image by automatic control of the image storage control module, and a user balance analysis module configured to perform a posture balance analysis of the user using the posture image stored in the image storage module. The posture analysis system described above is configured to photograph and analyze the user's original, uncorrected posture with the eyes closed, and thereby, has an effect of accurately analyzing postures of shoulders, pelvis, arms, legs, and the like of the user. In addition, the posture analysis system using a smart mirror and a camera provides feedback information for correction based on analysis results, and thereby, has an effect of continuously correcting posture through the smart mirror.

### CROSS-REFERENCE TO RELATED APPLICATION

This application claims priority to Korean Patent Application No. 10-2022-0128476 filed on Oct. 7, 2022, and all the benefits accruing therefrom under 35 U.S.C. § 119, the contents of which are incorporated by reference in their entirety.

### BACKGROUND

The present disclosure relates to a posture analysis system, and specifically to a posture analysis system using a smart mirror and a camera.

When checking for distortion of the shoulders or pelvis or checking the length of both legs or arms to correct posture, most users tend to consciously correct their posture, making accurate posture analysis difficult.

Since accurate diagnosis and prescriptions are possible only by analyzing daily unconscious posture, the diagnosis and prescriptions are useless when accurate posture analysis is not performed first.

Meanwhile, many hospitals and correction centers that perform posture correction, but it is not easy to continuously visit hospitals or correction centers to correct posture.

Therefore, there is a need for a means to accurately analyze and prescribe posture diagnosis and posture correction at any time in our daily living space.

Examples of the related art include Korean Patent Registration No. 10-1638004 and Korean Patent Laid-Open Publication No. 10-2016-0112703.

### SUMMARY

The present disclosure provides a posture analysis system using a smart mirror and a camera.

In accordance with an exemplary embodiment of the present disclosure, a posture analysis system using a smart mirror and a camera includes a smart mirror configured to reflect a posture of a user, a camera module configured to photograph the posture of the user and generate a posture image, a posture recognition module configured to recognize the posture image generated by the camera module, an image storage control module configured to automatically perform control to store the posture image, an image storage module configured to store the posture image by automatic control of the image storage control module, and a user balance analysis module configured to perform a posture balance analysis of the user using the posture image stored in the image storage module.

Here, the posture recognition module includes an object analyzer configured to recognize an object of the user in the posture image, a walking-in-place recognizer configured to recognize walking of the user in place based on a recognition result of the object analyzer, a neck movement recognizer configured to recognize neck movement of the user based on the recognition result of the object analyzer, and a pupil recognizer configured to recognize eye closure and eye opening of the user based on the recognition result of the object analyzer.

Further, the user balance analysis module includes a shoulder distortion analyzer configured to analyze presence and a degree of shoulder distortion of the user, a pelvic distortion analyzer configured to analyze presence and a degree of pelvic distortion of the user, a leg length analyzer configured to compare and analyze lengths of both legs of the user, and an arm length analyzer configured to compare and analyze lengths of both arms of the user.

Further, the posture analysis system further includes a feedback information database in which posture correction method information, lifestyle attitude information, and exercise information for posture correction according to the posture balance analysis of the user balance analysis module are stored, and a user balance analysis result feedback module configured to select the posture correction method information, the lifestyle attitude information, and the exercise information for posture correction stored in the feedback information database based on a result of the posture balance analysis of the user performed by the user balance analysis module and to feed back, to a user terminal, the result of the posture balance analysis, and the selected posture correction method information, lifestyle attitude information, and exercise information for posture correction.

Further, the posture analysis system further includes a hand gesture recognition module configured to recognize hand gestures of the user using the camera module, and a video guide output module configured to output a video guide for explaining actions for posture diagnosis according to a hand gesture recognized by the hand gesture recognition module through the smart mirror.

Further, the image storage control module is configured to perform control to start storing the posture image according to a hand gesture recognized by the hand gesture recognition module and to end storage of the posture image when the pupil recognizer recognizes the eye opening of the user.

Further, the posture analysis system further includes a microphone module configured to recognize a user command while the user is walking in place or making a neck movement, a re-recognition control module configured to control the image storage control module to cancel and restart storage of a posture image related to current walking in place or neck movement according to the user command recognized by the microphone module, and a speaker module configured to output a guide voice regarding control of the re-recognition control module.

### BRIEF DESCRIPTION OF THE DRAWINGS

Exemplary embodiments can be understood in more detail from the following description taken in conjunction with the accompanying drawings, in which:

FIG. 1 is a block diagram of a posture analysis system using a smart mirror and a camera in accordance with an exemplary embodiment of the present disclosure.

### DETAILED DESCRIPTION OF EMBODIMENTS

It is to be understood that the present disclosure may be variously modified and embodied, and thus particular exemplary embodiments thereof will be illustrated in the drawings and described in detail in the “Detailed Description of Embodiment” section. However, this is not intended to limit the present disclosure to the specific exemplary embodiments, it should be understood to include all modifications, equivalents, and substitutes included in the spirit and scope of the present disclosure. In describing each drawing, like reference numerals have been used for like elements.

It will be understood that, although the terms first, second, A, B, etc. may be used herein to describe various elements, these elements should not be limited by these terms. These terms are only used to distinguish one element from another element. For example, without departing from the scope of the present disclosure, a first element could be termed a second element, and similarly, a second element could be termed a first element. The term and/or includes a combination of a plurality of related listed items or any of a plurality of related listed items.

It will be understood that when an element is referred to as being “coupled” or “connected” to another element, the element may be directly coupled or connected to the other element, or intervening elements may also be present. In contrast, it will be understood that when an element is referred to as being “directly coupled” or “directly connected” to another element, there are not intervening elements present.

The terms used in the present application are merely provided to describe specific exemplary embodiments, and are not intended to limit the present disclosure. The singular forms, “a”, “an”, and “the” are intended to include the plural forms as well, unless the context clearly indicates otherwise. In the present application, it will be further understood that the terms “includes” and/or “having”, when used in this specification, specify the presence of stated features, numbers, steps, operations, elements, components, or combinations thereof, but do not preclude the presence or addition of one or more other features, numbers, steps, operations, elements, components, or combinations thereof.

Unless otherwise defined, all terms used herein, including technical or scientific terms, have the same meaning as commonly understood by those of ordinary skill in the art to which the exemplary embodiments of the present disclosure pertain. Terms, such as those defined in commonly used dictionaries, should be interpreted as having a meaning that is consistent with their meaning in the context of the related art and will not be interpreted in an idealized or overly formal sense unless expressly so defined herein.

Hereinafter, a preferred exemplary embodiment according to the present disclosure will be described in detail with reference to the accompanying drawings.

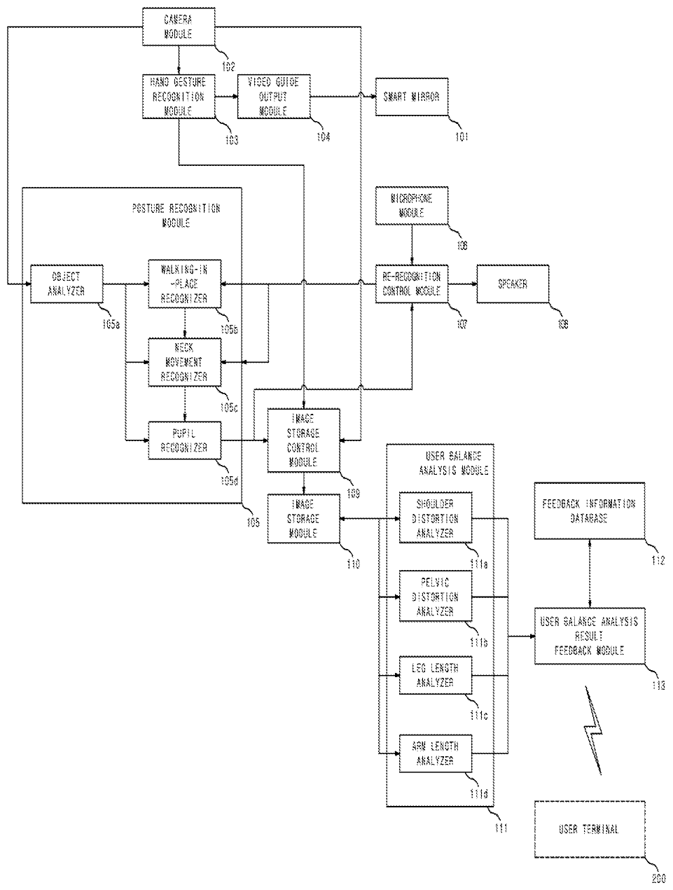

FIG. 1 is a block diagram of a posture analysis system using a smart mirror and a camera in accordance with an exemplary embodiment of the present disclosure.

Referring to FIG. 1, a posture analysis system 100 using a smart mirror and a camera in accordance with an exemplary embodiment of the present disclosure may include a smart mirror 101, a camera module 102, and a hand gesture recognition module 103, a video guide output module 104, a posture recognition module 105, a microphone module 106, a re-recognition control module 107, a speaker module 108, an image storage control module 109, an image storage module 110, a user balance analysis module 111, a feedback information database 112, and a user balance analysis result feedback module 113.

Hereinafter, the components will be described in detail.

The smart mirror 101 may be configured to reflect a posture of the user. The smart mirror 101 may reflect the posture of the user so that the posture is viewed with the naked eye.

The camera module 102 may be configured to photograph the posture of the user and generate a posture image. The camera module 102 may be configured to photograph the posture of the user in front of the smart mirror 101.

The hand gesture recognition module 103 may be configured to recognize hand gestures of the user using the camera module 102. The hand gesture recognition module 103 may have a motion recognition function and start a specific action or function using a pre-arranged hand gesture of the user.

The video guide output module 104 may be configured to output a video guide for explaining actions for posture diagnosis according to the hand gestures recognized by the hand gesture recognition module 103 through the smart mirror 101. A video for guiding the user on how to take a posture for posture diagnosis in front of the smart mirror 101 may be output.

The video guide may provide a guide to take postures, such as walking in place with eyes closed at least three times, closing the eyes and moving the head right and left more than three times, and closing the eyes and moving the head up and down more than three times. When opening the eyes, the user will see his or her posture on the smart mirror 101 and unconsciously correct the posture, and thus, the guide may guide the user to take the posture with the eyes closed.

The posture recognition module 105 may be configured to recognize the posture image generated by the camera module 102.

Detailed components of the posture recognition module 105, that is, an object analyzer 105a, a walking-in-place recognizer 105b, a neck movement recognizer 105c, and a pupil recognizer 105d, will be described below.

The object analyzer 105a may be configured to recognize an object of the user in the posture image. The object may be largely configured to recognize the overall shape and each joint, neck, and the like of the user.

The walking-in-place recognizer 105b may be configured to recognize walking of the user in place based on a recognition result of the object analyzer 105a. Walking in place may be recognized through the height of the knees and feet.

The neck movement recognizer 105c may be configured to recognize neck movement of the user based on the recognition result of the object analyzer 105a.

The pupil recognizer 105d may be configured to recognize eye closure and eye opening of the user based on the recognition result of the object analyzer 105a. The pupil recognizer 105d may be configured to perform the recognition by distinguishing between an eye open state and eye closed state depending on whether the pupil is recognized.

The microphone module 106 may be configured to recognize a user command while the user is walking in place or making a neck movement. For example, the user may give a user command to perform an action again.

The re-recognition control module 107 may be configured to control the image storage control module 109 to cancel and restart storage of a posture image related to current walking in place or neck movement according to the user command recognized by the microphone module 106.

The speaker module 108 may be configured to output a guide voice regarding control of the re-recognition control module 107. For example, the speaker module 108 may output a guide voice to resume walking in place.

The image storage control module 109 may automatically perform control to store the posture image.

Specifically, the image storage control module 109 may perform control to start storing a posture image according to a hand gesture recognized by the hand gesture recognition module 103 and to end storage of the posture image when the pupil recognizer 105d recognizes the eye opening of the user.

The image storage module 110 may be configured to store the posture image by automatic control of the image storage control module 109.

The user balance analysis module 111 may be configured to perform a posture balance analysis of the user using the posture image stored in the image storage module 110.

The user balance analysis module 111 may be configured to include a shoulder distortion analyzer 111a, a pelvic distortion analyzer 111b, a leg length analyzer 111c, and an arm length analyzer 111d.

Hereinafter, the components will be described in detail.

The shoulder distortion analyzer 111a may be configured to analyze presence and the degree of shoulder distortion of the user.

The pelvic distortion analyzer 111b may be configured to analyze presence and the degree of pelvic distortion of the user.

The leg length analyzer 111c may be configured to compare and analyze lengths of both legs of the user.

The arm length analyzer 111d may be configured to compare and analyze lengths of both arms of the user.

The feedback information database 112 may be configured so that posture correction method information, lifestyle attitude information, and exercise information for posture correction according to the posture balance analysis of the user balance analysis module 111 are stored therein.

The user balance analysis result feedback module 113 may be configured to select the posture correction method information, the lifestyle attitude information, and the exercise information for posture correction stored in the feedback information database 112 based on a result of the posture balance analysis of the user performed by the user balance analysis module 111 and to feed back, to a user terminal 200, the result of the posture balance analysis, and the selected posture correction method information, lifestyle attitude information, and exercise information for posture correction.

The user may perform correction training in front of the smart mirror 101 using corresponding feedback information.

The user balance analysis result feedback module 113 may recommend medical treatment when the result of the posture balance analysis of the user balance analysis module 111 does not meet a standard value.

The posture analysis system using a smart mirror and a camera described above is configured to photograph and analyze a user's original, uncorrected posture with the eyes closed, and thereby, has an effect of accurately analyzing postures of shoulders, pelvis, arms, legs, and the like of the user.

In addition, the posture analysis system provides feedback information for correction based on analysis results, and thereby, has an effect of continuously correcting posture through the smart mirror.

Although the present disclosure has been described with reference to the exemplary embodiment, it is to be understood that one ordinary skilled in the art can make various changes and modifications to the present disclosure without departing from the spirit and scope of the present disclosure as hereinafter claimed.

What is claimed is:

1. A posture analysis system using a smart mirror and a camera, the posture analysis system comprising:
a smart mirror (101) configured to reflect a posture of a user;
a camera module (102) configured to photograph the posture of the user and generate a posture image;
a posture recognition module (105) configured to recognize the posture image generated by the camera module (102);
an image storage control module (109) configured to automatically perform control to store the posture image;
an image storage module (110) configured to store the posture image by automatic control of the image storage control module (109); and
a user balance analysis module (111) configured to perform a posture balance analysis of the user using the posture image stored in the image storage module (105),
wherein the posture recognition module (105) comprises:
an object analyzer (105a) configured to recognize an object of the user in the posture image;
a walking-in-place recognizer (105b) configured to recognize walking of the user in place based on a recognition result of the object analyzer (105a);
a neck movement recognizer (105c) configured to recognize neck movement of the user based on the recognition result of the object analyzer (105a); and
a pupil recognizer (105d) configured to recognize eye closure and eye opening of the user based on the recognition result of the object analyzer (105a),
wherein the user balance analysis module (111) comprises:
a shoulder distortion analyzer (111a) configured to analyze presence and a degree of shoulder distortion of the user;
a pelvic distortion analyzer (111b) configured to analyze presence and a degree of pelvic distortion of the user;
a leg length analyzer (111c) configured to compare and analyze lengths of both legs of the user; and
an arm length analyzer (111d) configured to compare and analyze lengths of both arms of the user,
wherein the posture analysis system further comprises:
a feedback information database (112) in which posture correction method information, lifestyle attitude information, and exercise information for posture correction according to the posture balance analysis of the user balance analysis module (111) are stored; and
a user balance analysis result feedback module (113) configured to select the posture correction method information, the lifestyle attitude information, and the exercise information for posture correction stored in the feedback information database (112) based on a result of the posture balance analysis of the user performed by the user balance analysis module (111) and to feed back, to a user terminal (200), the result of the posture balance analysis, the posture correction method information, the lifestyle attitude information, and the exercise information for posture correction,
wherein the posture analysis system further comprises:
a hand gesture recognition module (103) configured to recognize hand gestures of the user using the camera module (102); and
a video guide output module (104) configured to output a video guide for explaining actions for posture diagnosis according to a hand gesture recognized by the hand gesture recognition module (103) through the smart mirror (101),
wherein the image storage control module (109) is configured to perform control to start storing the posture image according to a hand gesture recognized by the hand gesture recognition module (103) and to end storage of the posture image when the pupil recognizer (105d) recognizes the eye opening of the user,
wherein the posture analysis system further comprises:
a microphone module (106) configured to recognize a user command while the user is walking in place or making a neck movement;
a re-recognition control module (107) configured to control the image storage control module (109) to cancel and restart storage of a posture image related to current walking in place or neck movement according to the user command recognized by the microphone module (106); and
a speaker module (108) configured to output a guide voice regarding control of the re-recognition control module (107),
wherein the video guide provides a guide to take postures of walking in place with eyes closed at least three times, closing the eyes and moving a head right and left more than three times, and closing the eyes and moving the head up and down more than three times,
wherein the walking-in-place recognizer (105b) recognizes walking in place through a height of knees and feet, and
wherein the user balance analysis result feedback module (113) recommends medical treatment when the result of the posture balance analysis of the user balance analysis module (111) does not meet a standard value.
